(19) United States
(12) Patent Application Publication
(10) Pub. No.: US 2024/0156634 A1
(43) Pub. Date: May 16, 2024

(54) METHOD AND SUPPORT FOR ESTABLISHING ORAL RESPIRATORY AIRFLOW CHANNEL

(71) Applicant: Jun WANG, Huangshan city, Anhui province (CN)

(72) Inventor: Jun WANG, Huangshan city, Anhui province (CN)

(21) Appl. No.: 18/281,969

(22) PCT Filed: Jan. 24, 2022
(86) PCT No.: PCT/CN2022/000009

§ 371 Date: Sep. 14, 2023

(30) Foreign Application Priority Data
Mar. 26, 2021 (CN) 202110324291.X

(51) Int. Cl. A61F 5/56

(52) U.S. Cl. CPC A61F 5/566

(57) ABSTRACT

Disclosed is a support for establishing an oral respiratory airflow channel for treatment of sleep-disordered breathing. Two sides of the U-shape support are used as a left support body and a right support body. The left support body and the right support body are placed in a left oral vestibule and a right oral vestibule respectively. Therefore, a first respiratory airflow channel, which is distributed from a space between a maxillary tubercle and a retromolar region to the cavity (1) along outer peripheries of dentitions and gingivas via a pterygomandibular space, and a second respiratory airflow channel, which is formed by tiny spaces between an upper dentition masticatory surface and a lower dentition masticatory surface and enables communication between the proper oral cavity and an oral vestibule, jointly form the oral respiratory airflow channel.

### TECHNICAL FIELD

The present disclosure relates to health care facilities, and in particular to a method and support for establishing an oral respiratory airflow channel for treatment of sleep disordered nasal respiration.

### BACKGROUND

Oral respiration serves as a direct and effective means for treatment of sleep-disordered breathing.

All oral respiration aid devices currently commercially available in the market use the same principle. That is, a proper oral cavity is forced to change from a natural closed state to a semi-opened state, so as to establish an oral respiratory airflow channel from a throat to the outside of lips along an anteroposterior diameter of the proper oral. An exemplary technical method is shown in FIG. 1. A horseshoe-shaped pad is sandwiched between an upper tooth masticatory surface and a lower tooth masticatory surface. Upper teeth and lower teeth occlude with the pad sandwiched. One or two through holes are formed in the middle of a pad region between upper incisors and lower incisors in a front-and-back oral direction as a respiratory airflow access. Because of a sufficient thickness of the pad, an upper lip and a lower lip are also in an opened state, and therefore an oral respiratory airflow channel is established.

The pad in the above technology is convenient to wear and offers a desirable respiratory effect when in normal use. However, it still has the disadvantages as follows: an upwards-folded edge and a downwards-folded edge of the pad, along an inner periphery and an outer periphery of a dentition for locating, are formed, leading to an obvious foreign body sensation when in contact with the upper teeth, the lower teeth, a gingiva, and a tongue. Moreover, owing to physiological individual differences, a wearer may have an uncomfortable feeling once a pressure between part of the folded edges and the teeth and the gingiva is excessively high. In sleep, under the impact of the foreign body sensation and the uncomfortable feeling, more unconscious movement of the tongue and a mandibular joint will cause the pad to shift or fall off and become ineffective. In view of that, an elastic binding band that fits over the head and extends along a left cheek and a right cheek to be tied at a chin is offered in the above technology to limit the movement of the mandibular joint. However, since it fails to limit the movement of the tongue, the pad will still shift or fall off. Moreover, by virtue of a pulling force of the binding band, the upper teeth and the lower teeth keep occluding tightly with the pad sandwiched, so that the comfort is further reduced, and a user can hardly wear the binding band for a long time. In addition, the pad is typically made of soft silicone rubber. Owing to composite occlusion of the upper incisors and the lower incisors, the pad region between them has to bear a great pressure, and therefore silicone rubber bitten through by the upper incisors and the lower incisors enters the respiratory airflow access, leading to respiration obstruction and partial or complete failure of the pad.

### SUMMARY

An objective of the present disclosure is to overcome the disadvantages in convenience, comfort, reliability, and durability in the background art, and provides a support that can stably and reliably establish an oral respiratory airflow channel and is convenient, simple, and comfortable to wear.

The present disclosure adopts the following technical solution: the support is made of a soft elastic material, and is in a horizontal state when worn and in a U-shape structure when viewed from top of the support. Two sides of the U-shape structure are used as a left support body and a right support body respectively, which are bilaterally symmetrical. A closure end of the U-shape structure faces a front oral portion, is positioned between an upper lip and a lower lip, and is a cavity (1). The cavity includes a left respiratory airflow access and a right respiratory airflow access. A left end and a right end of the cavity are connected to a front end of the left support body and a front end of the right support body respectively. The left support body and the right support body are placed in a left oral vestibule and a right oral vestibule respectively. An upper edge (5) and a lower edge (6) of the support body are positioned in an upper vestibular sulcus (that is, an upper labial buccal gingival sulcus) and a lower vestibular sulcus (that is, a lower labial buccal gingival sulcus) respectively. A rear edge (7) of the support body abuts against a rear oral vestibular wall at an outer rear portion of a mandibular ramus. Therefore, a cheek inner wall is laterally pushed away from outer peripheries of dentitions and gingivas within a support range of the support body. A pterygomandibular mucosal fold is pulled and stretched. Moreover, a mandibular joint is slightly stretched, and a space of 0.5 mm or more is formed between an upper dentition masticatory surface and a lower dentition masticatory surface. Accordingly, a first respiratory airflow channel, which is distributed from a space between a maxillary tubercle and a retromolar region to the cavity (1) in a posteroanterior direction along outer peripheries of dentitions and gingivas via a pterygomandibular space, and a second respiratory airflow channel, which is formed by tiny spaces between the upper dentition masticatory surface and the lower dentition masticatory surface and enables a proper oral cavity to be in communication with an oral vestibule, jointly form the oral respiratory airflow channel.

A vertical size and a horizontal size of a rear portion of the above support body are significantly greater than those of a front portion of the support body, and the support body is converged and transited through a slope or a cambered surface in the posteroanterior direction.

The above support body includes a vertical support (2), and the vertical support is placed in an anteroposterior oral direction and perpendicular to a horizontal plane. The vertical support forms a body of the support body and independently realizes a support range for a cheek inner wall and slight stretching of a mandibular joint.

An auxiliary support (3) is attached to the above vertical support and bulges from an outer side surface of the vertical support to further push the cheek inner wall towards an oral outer side, and therefore, the pterygomandibular mucosal fold is further pulled and stretched.

The above vertical support includes an elastic structure (8), and the elastic structure generates a support force primarily in a vertical direction and may be in an annular shape or a semi-annular shape with a forward opening.

A height, bulging from the outer side surface of the vertical support, of the above auxiliary support can be changed through deformation of the vertical support or through forward or backward bending or tilting of the auxiliary support.

Grooves (4) are formed in inner surface of portions where the left end and the right end of the above cavity (1) are connected to the front end of the left support body and the front end of the right support body respectively. Respiratory airflow passes through the grooves when the inner surfaces are attached to the outer peripheries of the dentitions or the gingivas.

The above soft elastic material may be a homogeneous material or an elastic metal material coated with a soft material in part or in all.

A slight foreign body sensation is caused after the support is placed because an oral vestibule has a wide potential space. The support either makes contact with a tongue or resists oral movement in a sheep state, realizing desirable comfort. The support does not have a stressed portion that may be damaged, and thus has good durability. The vertical size and the horizontal size of the rear portion of the support body are significantly greater than those of the front portion of the support body, and a resultant force of acting forces of the cheek inner wall on the support body is directed to a rear oral portion. Therefore, it is not required to provide other auxiliary facilities. The support has a desirable automatic restoring capacity and wearing reliability. A minimum flow channel section between a unilateral maxillary tubercle and the retromolar region is 3 mm×6 mm or more, and a minimum flow channel section between unilateral upper and lower dentition masticatory surfaces generated after the mandibular joint is slightly stretched is 0.5 mm×30 mm or more, so that a respiratory effect is equivalent to that of the prior art. In the case of a side-lying position, the auxiliary support at this side is dented under stress to cause partial retraction of the pterygomandibular mucosal fold. Accordingly, a respiratory capacity of the first respiratory airflow channel is weakened. It should be noted that in the case of the side-lying position, a soft palate ptosis that causes disordered nasal respiration is relieved, and nasal respiration can be partially or completely recovered. In conclusion, the concept of the present disclosure conforms to a natural oral physiological feature, and the provided method for establishing the oral respiratory airflow channel is rational and effective. The support has the desirable wearing convenience, comfort, reliability, and durability, and suitable for being worn by a user for a long time. Compared with the prior art, significant advancements have been made.

### BRIEF DESCRIPTION OF THE DRAWINGS

FIG. 1 is an oblique view of the prior art; and

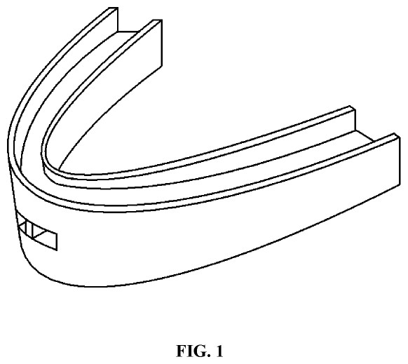

FIG. 2 is an oblique view of the present disclosure.

### DETAILED DESCRIPTION OF THE EMBODIMENTS

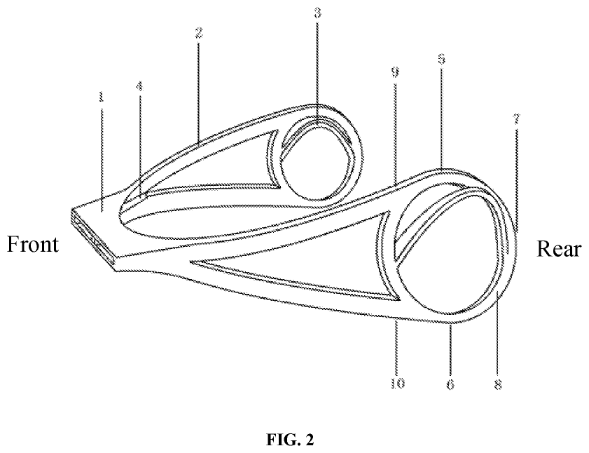

The particular implementation is described with reference to FIG. 2.

A support is made of homogeneous silicon rubber. A cavity (1) is internally provided with a left respiratory airflow access and a right respiratory airflow access. An upper surface and a lower surface of the cavity are attached to an upper lip and a lower lip respectively. A front edge of the cavity is flush with or slightly bulges from an outer edge of the lip. A distance between the upper surface and the lower surface of the cavity is 8 mm-10 mm. A vertical support (2) includes an annular elastic structure (8) and a V-shaped beam. The annular elastic structure is in an ellipse shape, a major axis of which is in a vertical direction, and a minor axis of which is in an anteroposterior direction. A distance between an upper edge (5) and a lower edge (6) of the annular elastic structure is 45 mm-50 mm, which is hierarchically determined according to individual differences. A rear end of the V-shaped beam is connected to the annular elastic structure. The V-shaped beam has the same thickness (6 mm-8 mm) as the annular elastic structure. A front end of the V-shaped beam is connected to the cavity (1). An auxiliary support (3) is in an arch shape. Two arch feet are attached to a front end and a rear end of an inner ring surface of the annular elastic structure respectively. A vertical height of an arch top is similar with an inner ring top height of the annular elastic structure. That is, an included angle between a plane where the auxiliary support is positioned and the vertical support is an acute angle. When the support is worn, the annular elastic structure is flattened in the vertical direction, the arch feet of the auxiliary support will generate moment to deflect the annular elastic structure outwards. In order to balance the moment, a rear side of the annular elastic structure deflects inwards by 10 degrees-15 degrees semi-around the ellipse major axis in the vertical direction. Grooves (4) are provided on inner surfaces of joints between the cavity (1) and the V-shape beam. All right-angle ridge lines in the figure are rounded.

Before the support is worn, a distance between inner surfaces of a left vertical support and a right vertical support is greater than that between a left oral vestibular sulcus and a right oral vestibular sulcus by 20 mm or more. That is, the support is worn through pre-tension, which depends on slight contact between the vertical supports and the gingivas after wearing. Pre-tension wearing is feasible in the presence of bilateral oral size differences of different wearers. A rear edge of the cavity (1) is close to positions of upper incisors and lower incisors and further dented forwards by 6 mm-8 mm on the basis of an original U-shaped radian, so that anteroposterior oral size differences of different wearers are compensated.

A method for wearing a support includes: positions (9) and (10) of the vertical supports on the same sides are pinched with thumbs and forefingers with the thumbs underneath of two hands respectively. The annular elastic structure is flattened in the vertical direction. In this case, the distance between the two arch feet of the auxiliary support is extended, and the arch top is lowered to be tightly attached to an outer side surface of the annular elastic structure. A mouth is opened moderately and does a grin action. Simultaneously, a rear edge (7) of the annular elastic structure is inserted in an oral vestibule from corners of the mouth and pushed towards a rear oral portion until the fingers touch the corners of the mouth. In this case, the support is released from two hands. The front edge of the cavity (1) is pressed with any finger to push an entire support into the oral cavity until the rear edge (7) of the annular elastic structure abuts against a rear oral vestibular wall at an outer rear portion of a mandibular ramus. Then the lips are closed, and the oral cavity is inflated to make a left oral vestibule and a right oral vestibule fully expand. Therefore, the arch top of the auxiliary support is stretched at an exact position, that is, wearing is completed.

A method for taking out a support includes: the mouth is opened moderately to expose the upper surface and the lower surface of the cavity (1). The upper surface and the lower surface of the cavity are pinched with the thumb and the forefinger with the thumb underneath of one hand. Cheek muscles are relaxed, and then the support is slowly pulled out. In this process, the annular elastic structure is deformed to reduce the vertical size, and the arch top of the auxiliary support is not completely dented.

1. A support for establishing an oral respiratory airflow channel, wherein the support is made of a soft elastic material, and is in a horizontal state when worn and in a U-shape structure when viewed from top of the support; two sides of the U-shape structure are used as a left support body and a right support body respectively, which are bilaterally symmetrical; a closure end of the U-shape structure faces a front oral portion, is positioned between an upper lip and a lower lip, and is a cavity (1), wherein the cavity comprises a left respiratory airflow access and a right respiratory airflow access; a left end and a right end of the cavity are connected to a front end of the left support body and a front end of the right support body respectively; the left support body and the right support body are placed in a left oral vestibule and a right oral vestibule respectively, an upper edge (5) and a lower edge (6) of the support body are positioned in an upper vestibular sulcus and a lower vestibular sulcus respectively, a rear edge (7) of the support body abuts against a rear oral vestibular wall at an outer rear portion of a mandibular ramus, thereby establishing a first respiratory airflow channel and a second respiratory airflow channel, wherein the first respiratory airflow channel is distributed from a space between a maxillary tubercle and a retromolar region to the cavity (1) in a posteroanterior direction along outer peripheries of dentitions and gingivas via a pterygomandibular space, and the second respiratory airflow channel is formed by tiny spaces between an upper dentition masticatory surface and a lower dentition masticatory surface and enables communication between a proper oral cavity and an oral vestibule, and the first respiratory airflow channel and the second respiratory airflow channel jointly form the oral respiratory airflow channel.

2. The support according to claim 1, wherein a vertical size and a horizontal size of a rear portion of the support body are greater than those of a front portion of the support body.

3. The support according to claim 1 or 2, wherein the support body comprises a vertical support (2), and the vertical support is placed in an anteroposterior oral direction and perpendicular to a horizontal plane.

4. The support according to claim 3, wherein an auxiliary support (3) is attached to the vertical support and bulges from an outer side surface of the vertical support.

5. The support according to claim 4, wherein a height, bulging from the outer side surface of the vertical support, of the auxiliary support (3) is changed through deformation of the vertical support (2).

6. The support according to claim 3, wherein the vertical support comprises an annular elastic structure (8), and the annular elastic structure generates a support force primarily in a vertical direction.

7. The support according to claim 3, wherein the vertical support comprises a semi-annular elastic structure (8), and the semi-annular elastic structure has a forward opening and generates a support force primarily in a vertical direction.

8. The support according to claim 1, wherein grooves (4) are formed in inner surfaces of portions where the left end and the right end of the cavity are connected to the front end of the left support body and the front end of the right support body respectively.

9. The support according to claim 1, wherein the soft elastic material is a homogeneous material.

10. The support according to claim 1, wherein the soft elastic material is an elastic metal material coated with a soft material.

11. The support according to claim 2, wherein the support body comprises a vertical support (2), and the vertical support is placed in an anteroposterior oral direction and perpendicular to a horizontal plane.

12. The support according to claim 11, wherein an auxiliary support (3) is attached to the vertical support and bulges from an outer side surface of the vertical support.

13. The support according to claim 12, wherein a height, bulging from the outer side surface of the vertical support, of the auxiliary support (3) is changed through deformation of the vertical support (2).

14. The support according to claim 11, wherein the vertical support comprises an annular elastic structure (8), and the annular elastic structure generates a support force primarily in a vertical direction.

15. The support according to claim 11, wherein the vertical support comprises a semi-annular elastic structure (8), and the semi-annular elastic structure has a forward opening and generates a support force primarily in a vertical direction.
